(19) United States
(12) Patent Application Publication
(10) Pub. No.: US 2020/0315455 A1
(43) Pub. Date: Oct. 8, 2020

(54) MEDICAL IMAGE PROCESSING SYSTEM AND METHOD FOR PERSONALIZED BRAIN DISEASE DIAGNOSIS AND STATUS DETERMINATION

(71) Applicant: Hyun Sub LEE, Seoul (KR)

(72) Inventors: Hyun Sub LEE, Seoul (KR); Kyung Mi LEE, Seoul (KR); Hyug Gi KIM, Seoul (KR)

(73) Assignee: Hyun Sub LEE, Seoul (KR)

(21) Appl. No.: 16/305,044

(22) PCT Filed: Feb. 23, 2018
(86) PCT No.: PCT/KR2018/002254

§ 371 Date: Nov. 27, 2018

(30) Foreign Application Priority Data
Apr. 4, 2017 (KR) 10-2017-0043734

(51) Int. Cl. A61B 5/00; A61B 5/026; A61B 5/02; G06T 7/00; G06T 11/00; G06T 7/11; G06T 7/62; G16H 30/40; G16H 50/20; G16H 50/30; G01R 33/563

(52) U.S. Cl. CPC A61B 5/0042; G06T 2207/30104; A61B 5/7275; A61B 5/7267; A61B 5/7282; A61B 5/02007; A61B 5/4082; A61B 5/4088; G06T 7/0012; G06T 11/003; G06T 7/11; G06T 7/62; G16H 30/40; G16H 50/20; G16H 50/30; G01R 33/5635; A61B 2576/026; G06T 2207/10088; G06T 2207/30016; A61B 5/0263

(57) ABSTRACT

A system for processing a medical image for personalized brain disease diagnosis and status determination, includes: an image processing unit, which obtains a 3D T1 weighted image, a 2D T2 fluid attenuated inversion recovery (FLAIR) image, a magnetic resonance angiogram (MRA) image, which images only a vessel for checking abnormality of a brain vessel, and a 4D phase-contrast flow image for recognizing a state of a blood flow in a vessel; a complex image analyzing unit, which selects a disease-to-be-diagnosed, sets a brain area according to the selected disease, and analyzes brain tissue and a brain vessel; and a personalized diagnosis and result output unit, which outputs a brain state, a disease-specific risk degree, a risk of disease, and a disease prediction result through a machine learning algorithm by utilizing an age-specific data DB.

### TECHNICAL FIELD

The present invention relates to a personalized brain disease diagnosis, and particularly, to a medical image processing system and method for personalized brain disease diagnosis and status determination, which are capable of predicting a current brain state, a disease-specific risk degree, and a disease by obtaining an image by using a noninvasive magnetic resonance image and analyzing composite images of brain tissue and a brain vessel.

### BACKGROUND ART

As society progresses, neurodegenerative diseases, such as Alzheimer diseases, become more common. The neurodegenerative disease is a brain-related disease, so that a method of diagnosing a disease without invasion is required.

As a method of diagnosing a noninvasive brain-related disease, an ultrasonic diagnosis is used.

The ultrasonic diagnosis is a carotid ultrasonic diagnosis, which is capable of noninvasively and easily diagnosing an atherosclerotic lesion of the carotid. Further, a cerebral blood flow within a cranial cavity is measured by a transcranial Doppler examination and is applied to clinic.

Another method includes a brain computerized tomography (CT), which is good to diagnose a hemorrhagic disease and is greatly helpful to treat a stroke patient by photographing a state of a brain blood flow and a brain vessel with a recently developed technology.

Further, a diagnosis method using a brain magnetic resonance imaging (MRI) is considered to be the best method for diagnosing a state of brain tissue. Compared to the brain CT, there is no influence of artifact by a skull, so that it is possible to minutely diagnose a lesion in the region of the brain stem, the cerebellum, the temporal lobe, early discover cerebral infarction and finely diagnose a state of brain perfusion, and thoroughly diagnose a state of the brain vessel.

The MRI is an imaging method of obtaining a predetermined cross-sectional image of a body by using a magnetic field generated by magnetic force. In the MRI, it is possible to obtain various images by adjusting an image variable, and an imaging method of brightening tissue having a short relaxation time is referred to as a T1 weighted imaging method and an imaging method of brightening tissue having a T2 long relaxation time is referred to as a T2 weighted imaging method. Further, it is possible to obtain a T2 weighted image, which is most appropriate for a molecular image and a cell image, by adjusting an image variable so that the tissue is brightly shown in the image when the T2 relaxation time is long.

However, when a brain disease is determined without invasion, there is a problem in that accuracy is degraded.

A brain disease analysis apparatus in the related art using a noninvasive diagnosis method is simply capable of accurately determining whether a patient to be diagnosed has a brain disease, but for a disability type and prognosis prediction of disability, the deduction of a meaning of a lesion observed in an MRI image mainly depends on a personal experience of a doctor.

Accordingly, there is a demand for development of a new technology, which is capable of specifying a brain lesion of a person to be diagnosed, determining a disability type based on a ratio of the brain lesion occupied based on a brain area, and predicting prognosis of the disability based on the degree of disability determined according to the ratio.

(Patent Document 1) Korean Patent Application Laid-Open No. 10-2015-0057045

(Patent Document 2) Korean Patent Application Laid-Open No. 10-2015-0135249

(Patent Document 3) Korean Patent Application Laid-Open No. 10-2014-0028534

### DISCLOSURE

### Technical Problem

The present invention is conceived to solve the problem of a brain disease diagnosis and medical image processing in the related art, and an object of the present invention is to provide a medical image processing system and method for personalized brain disease diagnosis and status determination, which are capable of predicting a current brain state, a disease-specific risk degree, and a disease by obtaining an image by using a noninvasive magnetic resonance image and analyzing composite images of brain tissue and a brain vessel.

Another object of the present invention is to provide a medical image processing system and method for personalized brain disease diagnosis and status determination, which are capable of outputting a personalized/disease-specific customized result through a diagnosis of composite images of brain tissue and a brain vessel by selecting a major disease, such as Alzheimer's dementia, Parkinson disease, cerebral stroke, and setting a brain area appropriate for the selected disease.

Another object of the present invention is to provide a medical image processing system and method for personalized brain disease diagnosis and status determination, which are capable of accurately performing a personalized/disease-specific analysis and diagnosis by utilizing age-specific customized data and applying a machine learning algorithm when complex images of brain tissue and a brain vessel are diagnosed.

Another object of the present invention is to provide a medical image processing system and method for personalized brain disease diagnosis and status determination, which efficiently perform an analysis and a diagnosis of complex images of brain tissue and a brain vessel by obtaining a 3D T1 weighted image and 2D T2 fluid attenuated inversion recovery (FLAIR) image by using a noninvasive magnetic resonance image.

Another object of the present invention is to provide a medical image processing system and method for personalized brain disease diagnosis and status determination, which are capable of accurately performing a personalized/disease-specific analysis and diagnosis by using a magnetic resonance angiogram (MRA) image, which images only a vessel for checking abnormality of a brain vessel, and a 4D phase-contrast flow image for recognizing a state of a blood flow in a vessel.

Another object of the present invention is to provide a medical image processing system and method for personalized brain disease diagnosis and status determination, which precisely predict a current brain state, a disease-specific risk degree, and a disease by outputting a volume of a disease-specific correlated brain with a quantitative numerical value and outputting a state of a small vessel of a brain with four small vessel levels (none, mild, moderate, and severe) through an analysis of brain tissue and a brain vessel.

The objects of the present invention are not limited to the foregoing objects, and those skilled in the art will clearly understand other non-mentioned objects through the description below.

### Technical Solution

In order to achieve the foregoing object, a medical image processing system for personalized brain disease diagnosis and status determination according to the present invention includes: an image processing unit, which obtains a 3D T1 weighted image, a 2D T2 fluid attenuated inversion recovery (FLAIR) image, a magnetic resonance angiogram (MRA) image, which images only a vessel for checking abnormality of a brain vessel, and a 4D phase-contrast flow image for recognizing a state of a blood flow in a vessel; a complex image analyzing unit, which selects a disease to be diagnosed, sets a brain area according to the selected disease, and analyzes brain tissue and a brain vessel; and a personalized diagnosis and result output unit, which outputs a brain state, a disease-specific risk degree, a risk of disease, and a disease prediction result through a machine learning algorithm by utilizing an age-specific data DB.

The image processing unit may include: an image receiving unit, which receives a non-invasive magnetic resonance image; a 3D T1 weighted image obtaining unit, which obtains a 3D T1 weighted image for checking a structural change of a brain and existence of functional abnormality according to the structural change of the brain through the image receiving unit; a 2D T2 FLAIR image obtaining unit, which obtains a 2D T2 FLAIR image for checking existence of abnormality in a small vessel reflected in brain tissue; a 3D MRA image obtaining unit, which obtains a 3D MRA image for checking a structural state of a large vessel and existence of abnormality according to the structural state; and a 4D phase-contrast flow image obtaining unit, which obtains a 4D phase-contrast flow image for digitizing the state of the blood flow in a large vessel with a visual and quantitative value and checks existence of abnormality in the vessel.

The complex image analyzing unit may include: a disease-to-be-diagnosed selecting unit, which selects a disease-to-be-diagnosed requiring a diagnosis; a brain area setting unit, which sets a brain area requiring an analysis according to the selected disease-to-be-diagnosed; a brain tissue analyzing unit, which measures a volume through the segmentation of the disease-specific related brain area by using the 3D T1 weighted image, and measures a volume through the segmentation of the area according to a brain function by using the 3D T1 weighted image; and a brain vessel analyzing unit, which performs a small vessel analysis through a WMH degree analysis using the 2D FLAIR image, a vessel tortuosity analysis using the 3D MRA image, and a large vessel analysis through an analysis of the state of the blood flow in the vessel using the 4D phase-contrast flow image.

The brain tissue analyzing unit may include: a brain structure centered analyzing unit, which measures the volume through the segmentation of the disease-specific related brain area by using the 3D T1 weighted image by using the fact that an atrophied brain area is different according to a disease, and analyzes the degree of progress of the atrophy of the brain area; and a brain function weighted analyzing unit, which measures a volume through the segmentation of the area of the 3D T1 weighted image according to the brain function, and analyzes the degree of progress of the atrophy.

The brain vessel analyzing unit may include: a small vessel analyzing unit, which automatically segments the WMH area of the brain by using the 2D FLIAR image, learns the volume and the number of clustering in stages, and classifies severity; and a large vessel analyzing unit, which makes the 3D MRA image into a maximum intensity projection (MIP) image, measures tortuosity of the large vessel by using the MIP image, and analyzes a state of the blood flow in the vessel by using the 4D phase-contrast flow image.

The personalized diagnosis and result output unit may include: a brain volume value output unit, which compares the volume with a DB of brains of an age group, which has similar related specific areas according to the selected disease, and outputs a measurement value of a volume of the area having a difference; a vessel level output unit, which compares the volume with a DB of brains of an age group, which has similar related specific areas according to the selected disease, and outputs a level of the vessel; and a disease-to-be-diagnosed state output unit, which outputs a structural change and a functional change of the brain with a numerical value according to the diagnosis and the analysis of the complex image analyzing unit.

The output of the personalized diagnosis result by the personalized diagnosis and result output unit may include displaying an image of a specific area related to the selected disease, and displaying brain information related to the selected disease together with the image.

The age-specific data DB may be established based on normal people, and be provided as reference data for a comparison and an analysis with a normal person when a specific disease is selected, and include a brain structure centered volume DB, which stores brain structure centered volume information, a brain function centered volume DB, which stores brain function centered volume information, a white matter hyperintensity (WMH) degree DB of a small vessel, which stores WMH information about a small vessel, a tortuosity DB of a large vessel, which stores information about tortuosity of a large vessel, and a blood flow state DB of a large vessel, which stores blood flow state information about a large vessel.

The disease-to-be-diagnosed may include Alzheimer's dementia disease, Parkinson disease, and cerebral stroke disease, and when the disease-to-be-diagnosed selecting unit selects a specific disease, cerebral nerve vessel automatic segmentation and brain anatomical area automatic segmentation related to the disease may be automatically performed.

In order to achieve another object, a method of processing a medical image for personalized brain disease diagnosis and status determination according to the present invention includes: when an image is input, obtaining a 3D T1 weighted image, a 2D T2 fluid attenuated inversion recovery (FLAIR) image, a 3D a magnetic resonance angiogram (MRA) image, and a 4D phase-contrast flow image; selecting a major disease and setting a brain area according to the selected disease; diagnosing and analyzing complex images of brain tissue and a brain vessel; performing a personalized diagnosis by utilizing a result of the diagnosis of the complex images of the brain tissue and the brain vessel and age-specific customized data; and outputting a brain state, a disease-specific risk degree, a risk of disease, and a disease prediction result according to the diagnosis and the analysis of the complex images of the brain tissue and the brain vessel.

In the analyzing of the complex images of the brain tissue and the brain vessel, the diagnosis and the analysis of the image of the brain tissue may include: a brain structure centered analysis, which measures a volume through segmentation of a disease-specific related brain area by using the 3D T1 weighted image by using the fact that an atrophied brain area is different depending on a disease, and analyzes the degree of progress of the atrophy of the brain area; and a brain function weighted analysis, which measures a volume through the segmentation of the area of the 3D T1 weighted image according to the brain function, and analyzes the degree of progress of the atrophy.

An analysis area for the brain structure centered analysis may include gray matter, white matter, and a cerebrospinal fluid (CSF).

The setting of the brain area in the brain function weighted analysis may include differently setting the brain area according to the selection of the disease including Alzheimer's dementia disease, Parkinson disease, and cerebral stroke disease.

In the diagnosing and the analyzing of the complex images of the brain tissue and the brain vessel, the diagnosis and the analysis of the image of the brain vessel may include: a small vessel analysis, which automatically segments the WMH area of the brain by using the 2D FLIAR image, learns the volume and the number of clustering in stages, and classifies severity; and a large vessel analysis, which makes the 3D MRA image into a maximum intensity projection (MIP) image, measures tortuosity of the large vessel by using the MIP image, and analyzes a blood flow state in the vessel by using the 4D phase-contrast flow image.

By the small vessel analysis, severity of a change in the white matter of the brain may be classified into stages of none, mild, moderate, and severe.

In the large vessel analysis, measurement areas are basilar artery (BA), left middle cerebra artery (MCA), and right MCA, and a pass length, a direct length, and a tortuosity value may be measured for each vessel, and a state of the vessel may be evaluated by using velocity, pressure, wall share stress, and a blood flow amount in the vessel.

In the large vessel analysis, the 3D MRA image may be obtained by imaging only a vessel for checking abnormality, such as an aneurysm, a vascular malformation, and a vascular form, of the brain vessel, and the 4D phase-contrast flow image may be obtained by continuously obtaining a 3D image according to a change in time, and a blood flow in the vessel is extracted with quantitative values of a speed (cm/sec), pressure (Pa), wall shear stress (N/m²), and a blood flow amount (ml/sec) through a post-processing process.

### Advantageous Effects

The medical image processing system and method for personalized brain disease diagnosis and status determination according to the present invention have the effects below.

First, it is possible to predict a current brain state, a disease-specific risk degree, and a disease by obtaining an image by using a noninvasive magnetic resonance image and analyzing complex images of brain tissue and a brain vessel.

Second, it is possible to output a personalized/disease-specific customized result through a diagnosis of composite images of brain tissue and a brain vessel by selecting a major disease, such as Alzheimer's dementia, Parkinson disease, cerebral stroke, and setting a brain area appropriate for the selected disease.

Third, it is possible to accurately perform a personalized/disease-specific analysis and diagnosis by utilizing age-specific customized data and applying a machine learning algorithm when complex images of brain tissue and a brain vessel are diagnosed.

Fourth, it is possible to efficiently analyze and diagnose complex images of brain tissue and a brain vessel by obtaining a 3D T1 weighted image and 2D T2 fluid attenuated inversion recovery (FLAIR) image.

Fifth, it is possible to accurately perform a personalized/disease-specific analysis and diagnosis by using a magnetic resonance angiogram (MRA) image, which images only a vessel for checking abnormality of a brain vessel, and a 4D phase-contrast flow image for recognizing a state of a blood flow in a vessel.

Sixth, it is possible to precisely predict a current brain state, a disease-specific risk degree, and a disease by outputting a volume of a disease-specific correlated brain with a quantitative numerical value and outputting a state of a small vessel of a brain with four small vessel levels (none, mild, moderate, and severe) through an analysis of brain tissue and a brain vessel.

### DESCRIPTION OF DRAWINGS

FIG. 1 is a diagram of a medical image processing system for personalized brain disease diagnosis and status determination according to the present invention.

FIG. 2 is a detailed diagram of an age-specific data DB.

FIG. 3 is a flowchart illustrating a medical image processing method for personalized brain disease diagnosis and status determination according to the present invention.

FIG. 4 is a detailed processing diagram of the medical image processing system for personalized brain disease diagnosis and status determination according to the present invention.

FIG. 5 is a related brain area classification table according to the kind of major disease.

FIG. 6 is a diagram of functional classification of a normal brain.

FIG. 7 is a diagram of detailed functional classification of a brain.

FIG. 8 is a diagram illustrating an analysis process of a brain vessel analyzing unit.

FIG. 9 is a diagram illustrating an analysis of a state of a blood flow in a vessel by using a 4D phase-contrast flow image.

FIG. 10 is a diagram illustrating an example of an output of a disease status of a person to be diagnosed.

### BEST MODE

A medical image processing system for personalized brain disease diagnosis and status determination according to the present invention includes: an image processing unit, which obtains a 3D T1 weighted image, a 2D T2 fluid attenuated inversion recovery (FLAIR) image, a magnetic resonance angiogram (MRA) image, which images only a vessel for checking abnormality of a brain vessel, and a 4D phase-contrast flow image for recognizing a state of a blood flow in a vessel; a complex image analyzing unit, which selects a disease-to-be-diagnosed, sets a brain area according to the selected disease, and analyzes brain tissue and a brain vessel; and a personalized diagnosis and result output unit, which outputs a brain state, a disease-specific risk degree, a risk of disease, and a disease prediction result through a machine learning algorithm by utilizing an age-specific data DB.

### MODE FOR CARRYING OUT THE INVENTION

Hereinafter, an exemplary embodiment of a medical image processing system and method for personalized brain disease diagnosis and status determination according to the present invention will be described in detail below.

Characteristics and advantages of the medical image processing system and method for personalized brain disease diagnosis and status determination according to the present invention will be apparent through the detailed description of each exemplary embodiment below.

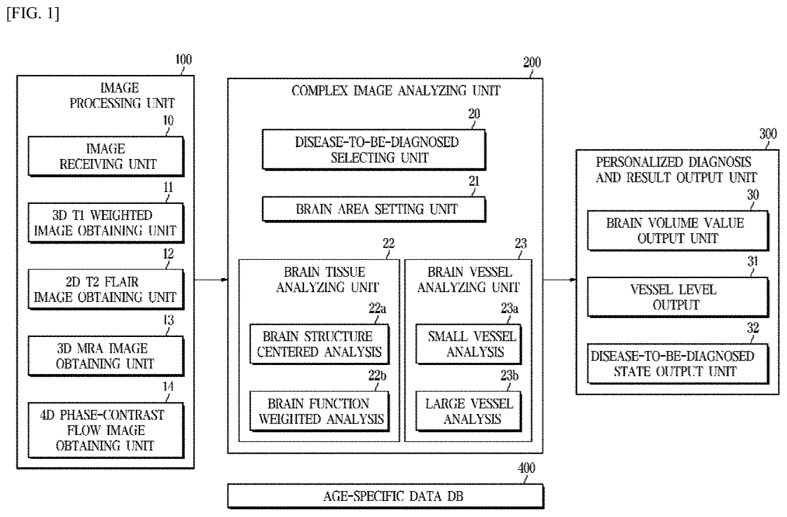

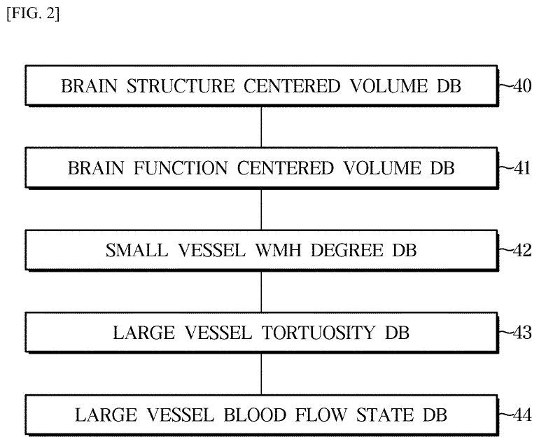

FIG. 1 is a diagram illustrating a medical image processing system for personalized brain disease diagnosis and status determination according to the present invention, and FIG. 2 is a detailed diagram of an age-specific data DB.

The medical image processing system and method for personalized brain disease diagnosis and status determination according to the present invention are capable of predicting a current brain state, a disease-specific risk degree, and a disease by obtaining an image by using a noninvasive magnetic resonance image and analyzing complex images of brain tissue and a brain vessel.

To this end, the present invention includes a configuration of selecting a major disease, such as Alzheimer's dementia, Parkinson disease, cerebral stroke, setting a brain area appropriate for the selected disease, and outputting a personalized/disease-specific customized result through a diagnosis of composite images of brain tissue and a brain vessel.

Further, the present invention includes a configuration of performing a personalized/disease-specific analysis and diagnosis by utilizing age-specific customized data and applying a machine learning algorithm when complex images of brain tissue and a brain vessel are diagnosed.

The present invention includes a configuration of performing an analysis and a diagnosis of complex images of brain tissue and a brain vessel by obtaining a 3D T1 weighted image and 2D T2 fluid attenuated inversion recovery (FLAIR) image.

The present invention includes a configuration of performing a personalized/disease-specific analysis and diagnosis by using a magnetic resonance angiogram (MRA) image, which images only a vessel for checking abnormality of a brain vessel, and a 4D phase-contrast flow image for recognizing a state of a blood flow in a vessel.

The present invention includes a configuration of outputting a volume of a brain having disease-specific correlation with a quantitative numerical value and outputting a state of a small vessel of a brain with four small vessel levels (none, mild, moderate, and severe) through an analysis of brain tissue and a brain vessel.

The medical image processing system and method for personalized brain disease diagnosis and status determination according to the present invention perform an analysis of complex images of brain tissue and a brain vessel, and includes a brain structure centered analysis, a brain function weighted analysis, a small vessel analysis, and a large vessel analysis.

The brain structure centered analysis method uses a fact that an atrophied brain area is different according to a disease, and automatically progresses segmentation of the areas of the brain related to a disease when a specific disease is selected, finds a brain area, which is more rapidly atrophied by the specific disease compared to a brain area of a normal person based on the segmentation, and indicates the degree of progress of the atrophy of a specific brain area with a state percentage (%).

Further, functions served according to an anatomical structure of a brain are divided into, for example, the areas of the frontal lobe (which is in charge of a body movement according to thought and determination), a parietal lobe (which is in charge of somatic sense information), a temporal lobe (which is in charge of a language function, auditory perception processing, and long-term memory and emotion), the occipital lobe (which is in charge of visual processing and recognition function), and the brain function weighted analysis method automatically segments the areas of the brain serving the functions of the brain and indicates the degree of progress of the atrophy of a brain area with a state percentage (%), compared to the same normal person.

Further, the small vessel analysis method classifies the degree of severity of a change in white matter of the brain into four levels, that is, none, mild, moderate, and severe.

The small vessel analysis method automatically segments a white matter hyperintensity (WMH) area of the brain by using a 2D FLIAR image, and then learns a volume and the number of clustering in stages, and classifies severity.

Further, the large vessel analysis method makes a 3D MRA image into a maximum intensity projection (MIP) image and measures tortuosity of a large vessel by using the MIP image.

The measurement areas are three in total, that is, the basilar artery (BA), the left middle cerebra artery (MCA), and the right MCA.

The large vessel analysis method measures a pass length, a direct length, and a tortuosity value for each vessel.

Further, the large vessel analysis method evaluates a state of a vessel by using velocity, pressure, wall share stress, and a blood flow amount in the vessel.

The medical image processing system for personalized brain disease diagnosis and status determination according to the present invention includes: an image processing unit 100, which obtains a 3D T1 weighted image, a 2D T2 FLAIR image, an MRA image, which images only a vessel for checking abnormality of a brain vessel, and a 4D phase-contrast flow image for recognizing a state of a blood flow in a vessel by using a non-invasive magnetic resonance image; a complex image analyzing unit 200, which selects a disease-to-be-diagnosed, sets a brain area according to the selected disease, and analyzes brain tissue and a brain vessel; and a personalized diagnosis and result output unit 300, which outputs a brain state, a disease-specific risk degree, a risk of disease, and a disease prediction result through a machine learning algorithm by utilizing an age-specific data DB 400, as illustrated in FIG. 1.

Herein, the image processing unit 100 includes an image receiving unit 10, which receives a non-invasive magnetic resonance image, a 3D T1 weighted image obtaining unit 11, which obtains a 3D T1 weighted image for checking a structural change of a brain and existence of functional abnormality according to the structural change of the brain through the image receiving unit 10, a 2D T2 FLAIR image obtaining unit 12, which obtains a 2D T2 FLAIR image for checking existence of abnormality in a small vessel reflected in brain tissue, a 3D MRA image obtaining unit 13, which obtains a 3D MRA image for checking a structural state of a large vessel and existence of abnormality according to the structural state, and a 4D phase-contrast flow image obtaining unit 14, which obtains a 4D phase-contrast flow image for digitizing the state of the blood flow in the large vessel with a visual and quantitative value and checking existence of abnormality in the vessel.

Further, the complex image analyzing unit 200 includes a disease-to-be-diagnosed selecting unit 20, which selects a disease-to-be-diagnosed requiring a diagnosis among the major diseases, such as Alzheimer's dementia disease, Parkinson disease, cerebral stroke disease, a brain area setting unit 21, which sets a brain area requiring an analysis according to the disease-to-be-diagnosed selected by the disease-to-be-diagnosed selecting unit 20, a brain tissue analyzing unit 22, which measures a volume through the segmentation of a disease-specific related brain area by using the 3D T1 weighted image, and measures a volume through the segmentation of the area according to a brain function by using the 3D T1 weighted image, and a brain vessel analyzing unit 23, which performs a small vessel analysis through a white matter hyperintensity analysis using the 2D FLAIR image, a vessel tortuosity analysis using the 3D MRA image, and a large vessel analysis through an analysis of a state of a blood flow in the vessel using the 4D phase-contrast flow image.

Herein, when the disease-to-be-diagnosed selecting unit 20 selects a specific disease, cerebral nerve and vessel automatic segmentation and brain anatomical area automatic segmentation related to the selected disease are performed.

Further, the brain tissue analyzing unit 22 includes a brain structure centered analyzing unit 22a, which finds a brain area, which is more rapidly atrophied by a specific disease compared to a brain area of a normal person, by measuring the volume through the segmentation of the disease-specific related brain area by using the 3D T1 weighted image by using the fact that an atrophied brain area is different according to a disease, and analyzes the degree of progress of the atrophy of a specific brain area, and a brain function weighted analyzing unit 22b, which measures a volume through the segmentation of the area of the 3D T1 weighed image according to the brain function, such as the frontal lobe (thought and determination), the temporal lobe (language, auditory sense, and long-term memory), the parietal lobe (perception and somatic sense information), and the occipital lobe (visual recognition), and analyzes the degree of progress of the atrophy of the brain area compared to the brain area of the normal person.

Further, the brain vessel analyzing unit 23 includes a small vessel analyzing unit 23a, which automatically segments the WMH area of the brain by using the 2D FLIAR image, learns the volume and the number of clustering in stages, and classifies severity, and a large vessel analyzing unit 23b, which makes the 3D MRA image into an MIP image, measures tortuosity of the large vessel by using the MIP image, and analyzes a state of the blood flow in the vessel by using the 4D phase-contrast flow image.

Further, the personalized diagnosis and result output unit 300 includes a brain volume value output unit 30, which compares the volume with a DB of the brains of an age group, which has the similar related specific area according to the selected disease, and outputs a measurement value of a volume of the area having a difference, a vessel level output unit 31, which compares the volume with a DB of the brains of an age group, which has the similar related specific area according to the selected disease, and outputs a level of the vessel, and a disease-to-be-diagnosed state output unit 32, which outputs a structural change and a functional change of the brain with a numerical value according to the diagnosis and the analysis of the complex image analyzing unit 200.

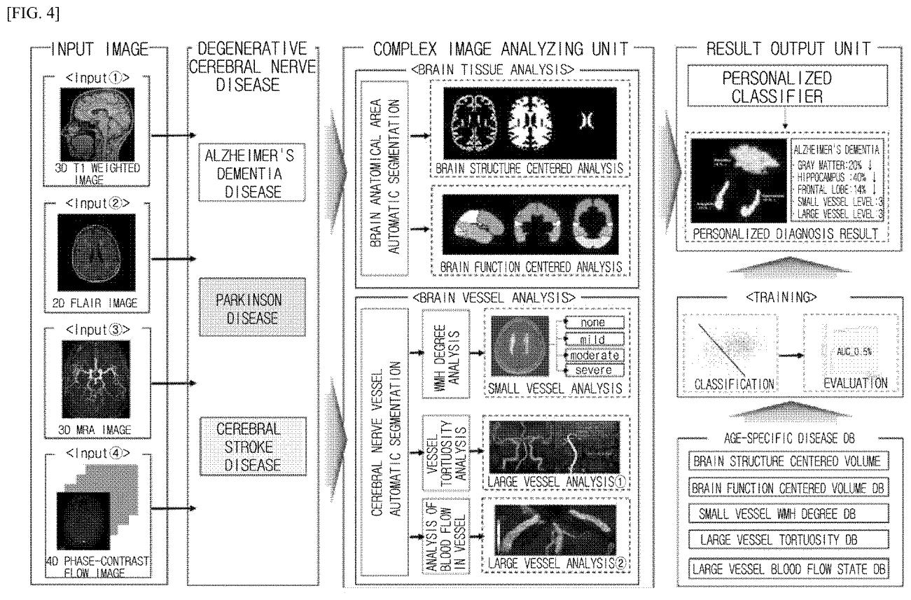

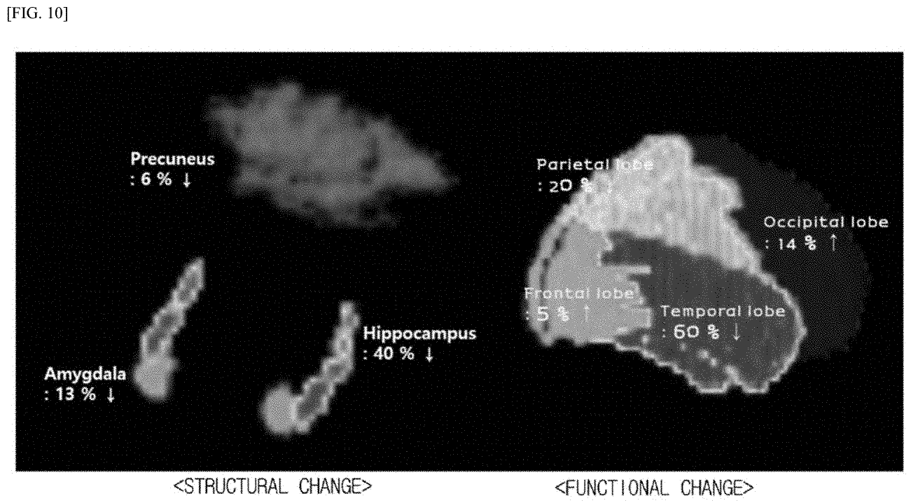

As an example of the output of the personalized diagnosis result by the personalized diagnosis and result output unit 300 including the foregoing configuration, an image of the specific area related to the selected disease (Alzheimer's dementia) is displayed, and information, such as [gray matter: 20% ↓], [hippocampus: 40% ↓], [frontal lobe: 14% ↓], [level of small vessel: 3], and [level of large vessel: 3], is displayed together with the image as illustrated in FIGS. 4 and 10.

The display of the image and the disease related brain information is varied according to the selected disease.

Further, the age-specific data DB 400 is established based on normal people, and is provided as reference data for the comparison and the analysis with a normal person when a specific disease is selected as illustrated in FIG. 2.

The configuration of the age-specific data DB 400 includes a brain structure centered volume DB 40, which stores brain structure centered volume information, a brain function centered volume DB 41, which stores brain function centered volume information, a WMH degree DB 42 of a small vessel, which stores WMH information about a small vessel, a tortuosity DB 43 of a large vessel, which stores information about tortuosity of a large vessel, and a blood flow state DB 44 of a large vessel, which stores blood flow state information about a large vessel.

A method of evaluating a standard deviation difference compared to an average value by using a generally used brain volume DB greatly depends on a numerical value of data.

In the method of evaluating a standard deviation difference, when data does not comply with a normal distribution chart, the evaluation of a difference based on a standard deviation is meaningless. When a standard deviation is large compared to an average value, the evaluation of a difference based on a standard deviation is actually statistically meaningless.

The method of evaluating a standard deviation difference does not consider the situation.

Further, when the features required for a diagnosis are numerous when a normal group and a disease group are classified, the classification is impossible. An average and a standard deviation are different according to each feature, so that it is difficult to evaluate a specific feature, which is to be used for the diagnosis.

The medical image processing system and method for personalized brain disease diagnosis and status determination according to the present invention perform the group classification by using machine learning in order to overcome the limit.

Among the group classification machine learning methods, a support vector machine classification method, that is a supervised learning method, is used.

As described above, when the SVM group classification method is used, an algorithm is a classification algorithm using a pattern-based distance ratio, so that it is possible to overcome a limit of an existing standard deviation evaluation method in analyzing a pattern of input data and automatically extract features having the highest classification accuracy from the multiple features.

The medical image processing method for personalized brain disease diagnosis and status determination according to the present invention will be described below.

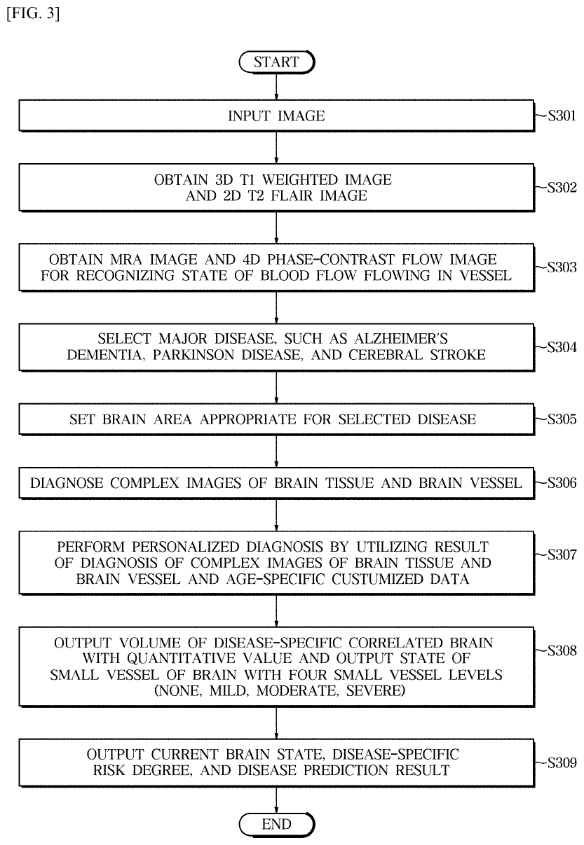

FIG. 3 is a flowchart illustrating the medical image processing method for personalized brain disease diagnosis and status determination according to the present invention.

As illustrated in FIG. 3, when an image is input (S301), a 3D T1 weighted image and a 2D T2 FLAIR image are obtained (S302).

Then, a 3D MRA image and a 4D phase-contrast flow image for recognizing a state of a blood flow in a vessel are obtained (S303).

The 3D MRA image is obtained by imaging only a vessel for checking abnormality, such as an aneurysm, a vascular malformation, and a vascular form, of a brain vessel.

Further, the 4D phase-contrast flow image is an image for recognizing a state of a blood flow in a vessel, and is obtained by continuously obtaining a 3D image according to a change in time. Then, the blood flow in the vessel is extracted with quantitative values of a speed (cm/sec), pressure (Pa), wall shear stress (N/m²), and a blood flow amount (ml/sec) through a post-processing process.

Next, the major disease, such as Alzheimer's dementia, Parkinson disease, and cerebral stroke, is selected (S304), and a brain area appropriate for the selected disease is set (S304).

Herein, the brain area appropriate for the selected disease may be set as described below.

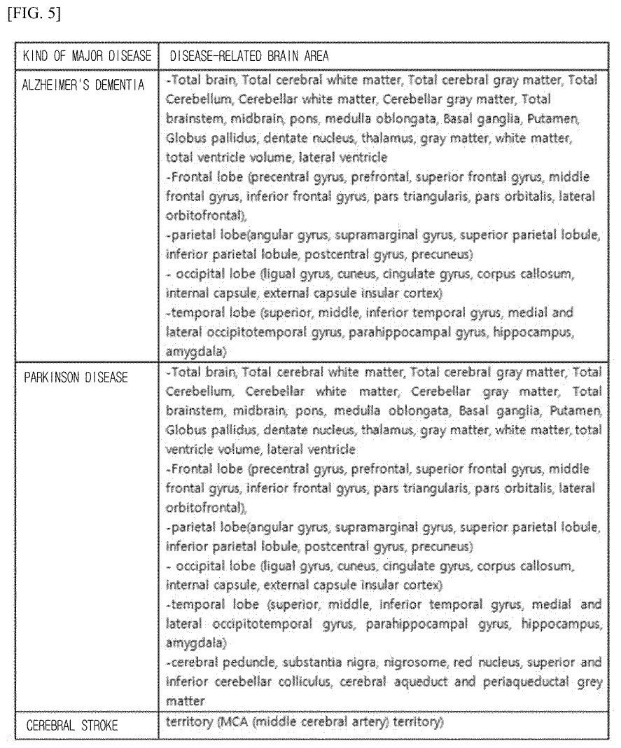

As illustrated in FIG. 5 illustrating a relevant brain area classification table according to the kind of major disease, the brain areas set when Alzheimer's dementia is selected are (total brain, Total cerebral white matter, Total cerebral gray matter, Total Cerebellum, Cerebellar white matter, Cerebellar gray matter, Total brainstem, midbrain, pons, medulla oblongata, Basal ganglia, Putamen, Globus pallidus, dentate nucleus, thalamus, Frontal lobe, parietal lobe, occipital lobe, temporal lobe, lobe-specific gray matter, lobe-specific white matter, total ventricle volume, lateral ventricle, temporal ventricle, frontal lobe (in detail, precentral gyrus, prefrontal, superior frontal gyrus, middle frontal gyrus, inferior frontal gyrus, pars triangularis, pars orbitalis, lateral orbitofrontal), parietal lobe (angular gyrus, supramarginal gyrus, superior parietal lobule, inferior parietal lobule, postcentral gyrus, precuneus), temporal lobe (superior, middle, inferior temporal gyrus, medial and lateral occipitotemporal gyrus, parahippocampal gyms, hippocampus, amygdala), occipital lobe (ligual gyrus, cuneus), cingulate gyms, corpus callosum, internal capsule, external capsule insular cortex).

Further, the brain areas set when Parkinson disease is selected are a dementia area+cerebral peduncle, substantia nigra, nigrosome, red nucleus, superior and inferior cerebellar colliculus, cerebral aqueduct and periaqueductal grey matter.

Further, the brain area set when cerebral stroke is selected is an artery-specific territory (middle cerebral artery (MCA) territory and the like).

Further, in the medical image processing method for personalized brain disease diagnosis and status determination according to the present invention, the functional classification of the brain is described below.

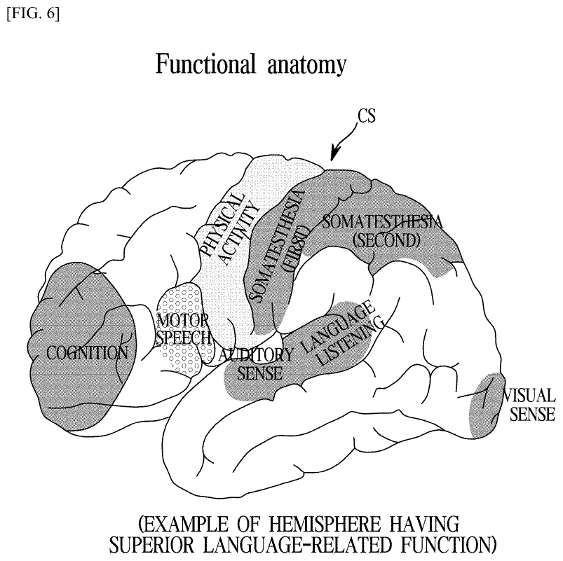

As illustrated in FIG. 6 illustrating the functional classification configuration of a normal brain, functional classification, such as a cognitive area, a motor speech area, a physical activity area, a somatesthesia area (first and second), an acoustic area, an acoustic language area, a language listening area, and a visual area, is used.

Further, the complex image analyzing unit 200 measures a volume through the segmentation of the disease-specific related brain area by using the 3D T1 weighted image, measures a volume through the area segmentation according to a brain function by using the 3D T1 weighted image, and performs a diagnosis of complex images of brain tissue and a brain vessel by performing a small vessel analysis through a WMH analysis using the 2D FLAIR image, a vessel tortuosity analysis using the 3D MRA image, and a large vessel analysis through an analysis of a state of the blood flow in the vessel by using the 4D phase-contrast flow image (S305).

Next, a personalized diagnosis is performed by using the result of the diagnosis of the complex images of the brain tissue and the brain vessel and age-specific customized data (S306).

Further, a volume of the brain having disease-specific correlation is output with a quantitative numerical value and a state of a small vessel of the brain is output with four small vessel levels (none, mild, moderate, and severe) (S307).

Further, a current brain state, a disease-specific risk degree, and a disease prediction result are output (S308).

The medical image processing method for personalized brain disease diagnosis and status determination according to the present invention will be described in detail based on each operation below.

FIG. 4 is a detailed processing diagram of the medical image processing system for personalized brain disease diagnosis and status determination according to the present invention.

The kind of image and a purpose of use of the image in the medical image processing system and method for personalized brain disease diagnosis and status determination according to the present invention are described below.

\textcircled{1} The 3D T1 weighted image is used for checking a change in a structure of the brain and existence of functional abnormality according to the change in the structure of the brain, \textcircled{2} the 2D T2 FLAIR image is used for checking existence of abnormality of a small vessel reflected in brain tissue, \textcircled{3} the 3D MRA image is used for checking a structural state of a large vessel and existence of abnormality according to the structural state of the large vessel, and \textcircled{4} the 4D phase-contrast flow image is used for digitizing a state of a blood flow in a large vessel with a visual and quantitative value and checking existence of abnormality of the vessel.

In the present invention, the analysis of the brain tissue is performed through a brain structure centered analysis and a brain function centered analysis.

The brain structure centered analysis is performed by measuring a volume through the segmentation of the disease-specific correlated brain area by using the 3D T1 weighted image, and the analysis areas are gray matter, white matter, and a cerebrospinal fluid (CSF).

Further, the brain areas related to the disease are hippocampus, precuneus, amygdala, and the like related to Alzheimer's dementia, putamen, substantia nigra, and the like related to Parkinson disease, and an area supplying an artery-specific vessel and the like related to cerebral stroke.

The analysis method uses a fact that an atrophied brain area is different according to a disease, and automatically progresses segmentation of the areas of the brain related to a disease when a specific disease is selected, finds a brain area, which is more rapidly atrophied by the specific disease compared to a brain area of a normal person based on the segmentation, and indicates the degree of progress of the atrophy of a specific brain area with a state percentage (%).

For example, personalized hippocampus atrophy degree (%)=a volume of hippocampus area (mm³)/entire brain area (mm³)×100.

A size of the hippocampus is different according to an individual, so that the personalized hippocampus atrophy degree is calculated and indicated with a relative percentage (%) based on the entire brain tissue area.

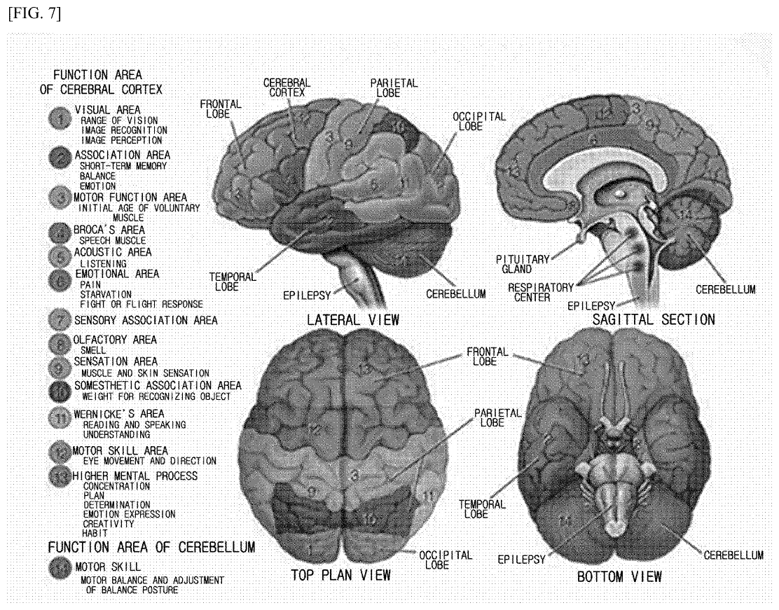

Further, the brain function centered analysis is performed by segmenting the area related to the brain function by using the 3D T1 weighted image, and the brain function areas are the frontal lobe (thought and determination), the temporal lobe (language, auditory sense, and long-term memory), the parietal lobe (perception and somatic sense information), and the occipital lobe (visual recognition) as illustrated in FIG. 7, and the volume of the brain function area is measured through the area segmentation according to the brain function.

The analysis method automatically segments the brain areas serving the brain functions by using the fact that the served functions are different according to the anatomical structure of the brain and indicates the degree of progress of the atrophy of the brain area with a relative percentage (%), compared to a normal person.

Further, the brain vessel is analyzed and evaluated through the small vessel analysis and the large vessel analysis.

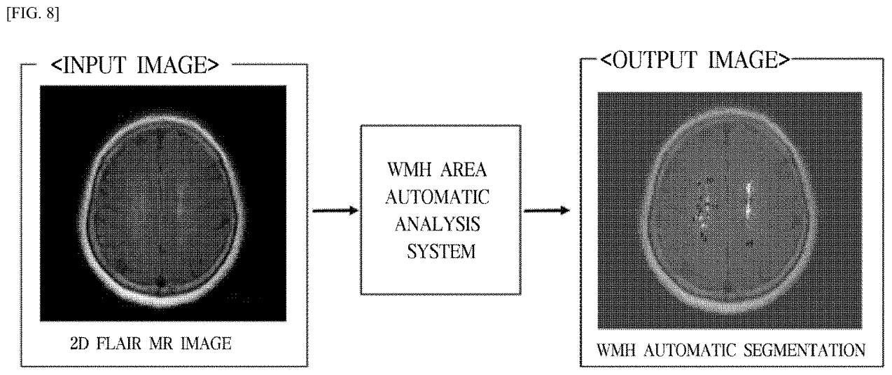

FIG. 8 is a diagram illustrating an analysis process of the brain vessel analyzing unit.

The small vessel analysis is performed through the WMH degree analysis by using the 2D FLAIR image, and the degree of severity of the change in the white matter is classified into four stages.

The four stages of the severity are none, mild, moderate, and severe.

The small vessel analysis automatically segments the WMH area of the brain by using the 2D FLIAR image, and then learns a volume and the number of clustering in stages, and classifies severity.

Further, the large vessel analysis is performed through the analysis of the degree of tortuosity of the vessel by using the 3D MRA image.

The large vessel analysis makes the 3D MRA image into an MIP image and measures tortuosity of the large vessel by using the MIP image.

The measurement areas are three in total, that is, the BA, the left MCA, and the right MCA. The large vessel analysis measures a pass length, a direct length, and a tortuosity value for each vessel.

Further, the analysis of the state of the blood flow in the vessel by using the 4D phase-contrast flow image is performed as described below.

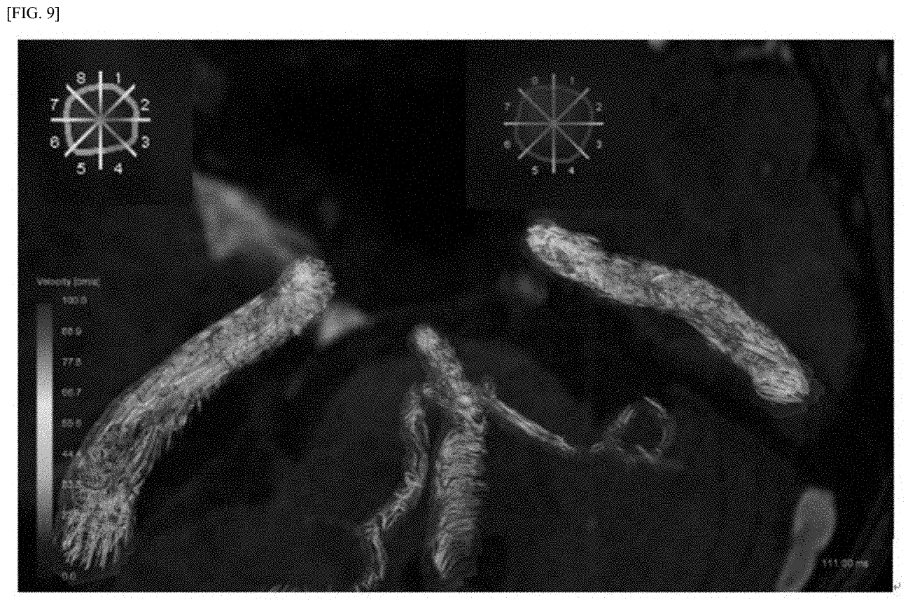

FIG. 9 is a diagram illustrating the analysis of the state of the blood flow in the vessel by using the 4D phase-contrast flow image.

The 4D phase-contrast flow image is an image for recognizing a state of a blood flow in a vessel, and a 3D image is continuously obtained according to a change in time.

Then, factors, based on which the state of the blood flow in the vessel are to be evaluated, are extracted through a post-processing process. As a result, velocity (cm/sec), pressure (Pa), wall share stress (N/m²), and a blood flow amount (ml/sec) in the vessel may be extracted with quantitative values.

The medical image processing system and method for personalized brain disease diagnosis and status determination may output a personalized/disease-specific customized result through a diagnosis of composite images of brain tissue and a brain vessel by selecting a major disease, such as Alzheimer's dementia, Parkinson disease, cerebral stroke, and setting a brain area appropriate for the selected disease.

Further, the present invention may accurately perform a personalized/disease-specific analysis and diagnosis by utilizing age-specific customized data and applying a machine learning algorithm when complex images of brain tissue and a brain vessel are diagnosed.

As described above, it may be appreciated that the present invention is implemented in the form modified without departing from the essential characteristic of the present invention.

Therefore, the exemplary embodiments shall be considered in an illustrative point of view, not a restrictive point of view, the scope of the present invention is illustrated in the claims, not the foregoing description, and it shall be appreciated that all of the differences within the equivalent range of the claim are included in the present invention.

1. A system for processing a medical image for personalized brain disease diagnosis and status determination, the system comprising:
an image processing unit, which obtains a 3D T1 weighted image, a 2D T2 fluid attenuated inversion recovery (FLAIR) image, a magnetic resonance angiogram (MRA) image, which images only a vessel for checking abnormality of a brain vessel, and a 4D phase-contrast flow image for recognizing a state of a blood flow in a vessel;
a complex image analyzing unit, which selects a disease-to-be-diagnosed, sets a brain area according to the selected disease, and analyzes brain tissue and a brain vessel; and
a personalized diagnosis and result output unit, which outputs a brain state, a disease-specific risk degree, a risk of disease, and a disease prediction result through a machine learning algorithm by utilizing an age-specific data DB,
wherein the age-specific data DB is established based on normal people, and is provided as reference data for a comparison and an analysis with a normal person when a specific disease is selected, and includes a brain structure centered volume DB, which stores brain structure centered volume information, a brain function centered volume DB, which stores brain function centered volume information, a white matter hyperintensity (WMH) degree DB of a small vessel, which stores WMH information about a small vessel, a tortuosity DB of a large vessel, which stores information about tortuosity of a large vessel, and a blood flow state DB of a large vessel, which stores blood flow state information about a large vessel.

2. The system of claim 1, wherein the image processing unit includes:
an image receiving unit, which receives a non-invasive magnetic resonance image;
a 3D T1 weighted image obtaining unit, which obtains a 3D T1 weighted image for checking a structural change of a brain and existence of functional abnormality according to the structural change of the brain through the image receiving unit;
a 2D T2 FLAIR image obtaining unit, which obtains a 2D T2 FLAIR image for checking existence of abnormality in a small vessel reflected in brain tissue;
a 3D MRA image obtaining unit, which obtains a 3D MRA image for checking a structural state of a large vessel and existence of abnormality according to the structural state; and
a 4D phase-contrast flow image obtaining unit, which obtains a 4D phase-contrast flow image for digitizing the state of the blood flow in a large vessel with a visual and quantitative value and checks existence of abnormality in the vessel.

3. The system of claim 1, wherein the complex image analyzing unit includes:
a disease-to-be-diagnosed selecting unit, which selects a disease-to-be-diagnosed requiring a diagnosis;
a brain area setting unit, which sets a brain area requiring an analysis according to the selected disease-to-be-diagnosed;
a brain tissue analyzing unit, which measures a volume through the segmentation of the disease-specific related brain area by using the 3D T1 weighted image, and measures a volume through the segmentation of the area according to a brain function by using the 3D T1 weighted image; and
a brain vessel analyzing unit, which performs a small vessel analysis through a WMH degree analysis using the 2D FLAIR image, a vessel tortuosity analysis using the 3D MRA image, and a large vessel analysis through an analysis of the state of the blood flow in the vessel using the 4D phase-contrast flow image.

4. The system of claim 3, wherein the brain tissue analyzing unit includes:
a brain structure centered analyzing unit, which measures the volume through the segmentation of the disease-specific related brain area by using the 3D T1 weighted image by using the fact that an atrophied brain area is different according to a disease, and analyzes the degree of progress of the atrophy of the brain area; and
a brain function weighted analyzing unit, which measures a volume through the segmentation of the area of the 3D T1 weighted image according to the brain function, and analyzes the degree of progress of the atrophy.

5. The system of claim 3, wherein the brain vessel analyzing unit includes:
a small vessel analyzing unit, which automatically segments the WMH area of the brain by using the 2D FLIAR image, learns the volume and the number of clustering in stages, and classifies severity; and
a large vessel analyzing unit, which makes the 3D MRA image into a maximum intensity projection (MIP) image, measures tortuosity of the large vessel by using the MIP image, and analyzes a state of the blood flow in the vessel by using the 4D phase-contrast flow image.

6. The system of claim 1, wherein the personalized diagnosis and result output unit includes:
a brain volume value output unit, which compares the volume with a DB of brains of an age group, which has similar related specific areas according to the selected disease, and outputs a measurement value of a volume of the area having a difference;
a vessel level output unit, which compares the volume with a DB of brains of an age group, which has similar related specific areas according to the selected disease, and outputs a level of the vessel; and
a disease-to-be-diagnosed state output unit, which outputs a structural change and a functional change of the brain with a numerical value according to the diagnosis and the analysis of the complex image analyzing unit.

7. The system of claim 6, wherein the output of the personalized diagnosis result by the personalized diagnosis and result output unit includes displaying an image of a specific area related to the selected disease, and displaying brain information related to the selected disease together with the image.

8. The system of claim 1, wherein the disease-to-be-diagnosed includes Alzheimer's dementia disease, Parkinson disease, and cerebral stroke disease, and
when the disease-to-be-diagnosed selecting unit selects a specific disease, cerebral nerve vessel automatic segmentation and brain anatomical area automatic segmentation related to the disease are automatically performed.

9. A method of processing a medical image for personalized brain disease diagnosis and status determination, the method comprising when an image is input, obtaining, by an image processing unit, a 3D T1 weighted image, a 2D T2 fluid attenuated inversion recovery (FLAIR) image, a 3D a magnetic resonance angiogram (MRA) image, and a 4D phase-contrast flow image;
selecting, by a complex image analyzing unit, a major disease, and setting a brain area according to the selected disease;
analyzing, by the complex image analyzing unit, complex images of brain tissue and a brain vessel of the set brain area; and
outputting, by the complex image analyzing unit, a brain state, a disease-specific risk degree, a risk of disease, and a disease prediction result through a machine learning algorithm by utilizing a result of the analysis of the brain tissue and the brain vessel and an age-specific data DB,
wherein the age-specific data DB is established based on normal people, and is provided as reference data for a comparison and an analysis with a normal person when a specific disease is selected, and includes a brain structure centered volume DB, which stores brain structure centered volume information, a brain function centered volume DB, which stores brain function centered volume information, a white matter hyperintensity (WMH) degree DB of a small vessel, which stores WMH information about a small vessel, a tortuosity DB of a large vessel, which stores information about tortuosity of a large vessel, and a blood flow state DB of a large vessel, which stores blood flow state information about a large vessel.

10. The method of claim 9, wherein in the analyzing of the complex images of the brain tissue and the brain vessel, the analysis of the image of the brain tissue includes:
a brain structure centered analysis, which measures a volume through segmentation of a disease-specific related brain area by using the 3D T1 weighted image by using the fact that an atrophied brain area is different according to a disease, and analyzes the degree of progress of the atrophy of the brain area; and
a brain function weighted analysis, which measures a volume through the segmentation of the area of the 3D T1 weighted image according to the brain function, and analyzes the degree of progress of the atrophy.

11. The method of claim 10, wherein an analysis area for the brain structure centered analysis includes gray matter, white matter, and a cerebrospinal fluid (CSF).

12. The method of claim 10, wherein the setting of the brain area in the brain function weighted analysis includes differently setting the brain area according to the selection of the disease including Alzheimer's dementia disease, Parkinson disease, and cerebral stroke disease.

13. The method of claim 9, wherein in the analyzing of the complex images of the brain tissue and the brain vessel, the analysis of the image of the brain vessel includes:
a small vessel analysis, which automatically segments the WMH area of the brain by using the 2D FLIAR image, learns the volume and the number of clustering in stages, and classifies severity; and
a large vessel analysis, which makes the 3D MRA image into a maximum intensity projection (MIP) image, measures tortuosity of the large vessel by using the MIP image, and analyzes a blood flow state in the vessel by using the 4D phase-contrast flow image.

14. The method of claim 13, wherein by the small vessel analysis, severity of a change in the white matter of the brain is classified into stages of none, mild, moderate, and severe.

15. The method of claim 13, wherein in the large vessel analysis, measurement areas are basilar artery (BA), left middle cerebra artery (MCA), and right MCA, and a pass length, a direct length, and a tortuosity value are measured for each vessel, and
a state of the vessel is evaluated by using velocity, pressure, wall share stress, and a blood flow amount in the vessel.

16. The method of claim 13, wherein in the large vessel analysis, the 3D MRA image is obtained by imaging only a vessel for checking abnormality, such as an aneurysm, a vascular malformation, and a vascular form, of the brain vessel, and
the 4D phase-contrast flow image is obtained by continuously obtaining a 3D image according to a change in time, and a blood flow in the vessel is extracted with quantitative values of a speed (cm/sec), pressure (Pa), wall shear stress (N/m2), and a blood flow amount (ml/sec) through a post-processing process.
